(19) United States
(12) Patent Application Publication
(10) Pub. No.: US 2004/0116352 A1
(43) Pub. Date: Jun. 17, 2004

(54) Active substance combination containing a compound with an opioid effect and at least one further compound of formula 1

(76) Inventors: Boris Chizh, Cambridge (GB); Thomas Christoph, Aachen (DE); Babette-Yvonne Koegel, Langerwehe-Hamich (DE); Achim Schuettler, Aachen (DE); Oswald Zimmer, Wuerselen (DE)

Correspondence Address: PERMAN & GREEN, 425 POST ROAD, FAIRFIELD, CT, 06824, US

(21) Appl. No.: 10/296,521

(22) PCT Filed: May 10, 2001
(86) PCT No.: PCT/EP01/05346

(30) Foreign Application Priority Data
May 26, 2000 (DE) 100-25-948.0

(51) Int. Cl. A61K 38/04; A61K 31/485

(52) U.S. Cl. 514/19; 514/282

(57) ABSTRACT

The invention relates to an active substance combination that contains a compound with an opioid effect and at least one further compound of the general formula (I) and/or one of its diastereomers and/or one of its enantiomers and/or at least one corresponding physiologically acceptable salt thereof. The invention further relates to medicaments that contain the inventive active substance combination, to pharmaceutical formulations that contain the active substance combination and to the use of the active substance combination for producing a medicament.

The present invention relates to an active substance combination containing a compound with an opioid effect and at least one further compound of the general formula I and/or one of its diastereomers and/or one of its enantiomers and/or at least one corresponding physiologically acceptable salt, medicaments containing this active substance combination, pharmaceutical formulations containing the active substance combination and the use of the active substance combination for the manufacture of a medicament.

Pain is one of the basic symptoms in clinical medicine. There is a worldwide requirement for effective pain therapy. The urgent requirement for patient- and target-orientated treatment for chronic and non-chronic pain, which should also be understood to mean successful and satisfactory pain treatment for patients, is documented in the high number of scientific works in the field of applied analgesics or basic research into nociception which have recently appeared.

Conventional opioids, such as morphine, for example, are effective in the treatment of severe to extremely severe pain, but have undesirable side effects, such as respiratory depression, nausea, vomiting, dependence, sedation, constipation or tolerance development.

The object on which the invention is based consists, therefore, in providing analgesically effective medicaments suitable for the treatment of severe to extremely severe pain. In addition, these medicaments should have as few as possible of the side effects of the known opioid analgesics, such as respiratory depression, nausea, vomiting, dependence, sedation, constipation or tolerance development.

According to the invention, this object is achieved by the provision of a new active substance combination containing

A) a compound with an opioid effect and/or at least one of its physiologically acceptable salts and

B) at least one compound of the general formula I,

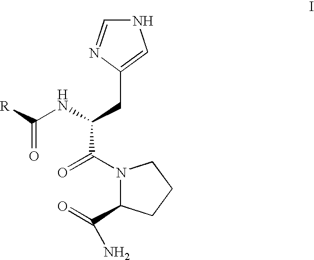

where the group R stands for one of the following groups a) to e)

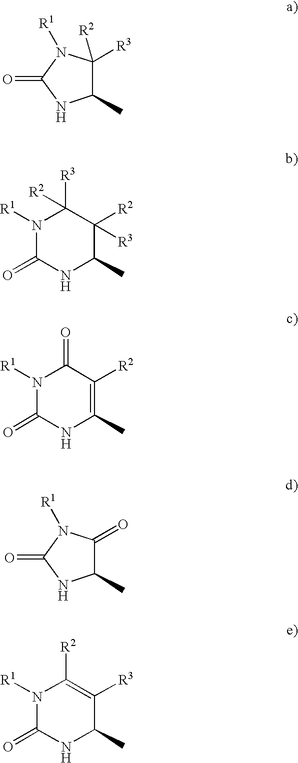

in which the groups R¹, R², R³, the same or different, mean H or a CH₃ group, and/or one of its enantiomers and/or one of its diastereomers and/or at least one corresponding physiologically acceptable salt.

Surprisingly, the active substance combination of the invention has a pronounced analgesic effect whereby the undesirable side effects, which are caused by the sole administration of compounds with an opioid effect, no longer occur or only occur in a very mild form.

The production of the compounds of the general formula I, its enantiomers, diastereomers and the production of the corresponding, physiologically acceptable salts may be performed in accordance with the disclosure in DE-PS-2449167, EP-0 429 245 or J. Med. Chem., 1990, 33(8), pages 2130 et seq by converting the corresponding carboxylic acids known to a person skilled in the art. The production of the compounds with an opioid effect and the corresponding salts is also known from literature, e.g. from E. Friderichs, T. Christoph, H. Buschmann, “Analgesics and Antipyretics”, Ullman's Encylopedia of Industrial Chemistry, Sixth Edition on CD-ROM, Wiley-VCH, Weinhem, 2000. The corresponding publications are hereby incorporated as references.

As a physiologically acceptable salt of the compounds of the general formula I and/or its enantiomers and/or diastereomers, there may advantageously be used hydrochloride, hydrobromide, sulphate, sulphonate, phosphate, tartrate, embonate, formate, acetate, propionate, benzoate, oxalate, succinate, citrate, glutamate, fumarate, aspartate, glutarate, stearate, butyrate, malonate, lactate, mesylate or a mixture of at least two of these salts.

The inventive active substance combination may contain the compounds of the general formula I, its diastereomers, enantiomers and its physiologically acceptable salts both individually and in the form of a mixture of at least two of these compounds. Preferably, the inventive active substance combination contains a compound of the general formula I, its enantiomers, its diasteromers or a corresponding physiologically acceptable salt as component B.

In a preferred embodiment of the invention, the active substance combination contains as component B) a compound of the general formula I, with the group R standing for the group a) and the groups R¹, R² and R³ meaning H, and/or one of its enantiomers and/or one of its diastereomers and/or one corresponding physiologically acceptable salt.

In another preferred embodiment of the invention, the inventive active substance combination contains as component B) a compound of the general formula I, with the group R standing for the group c) and R¹═CH₃ and R²═H, and/or one of its enantiomers and/or one of its diasteromers and/or a corresponding physiologically acceptable salt.

As a compound with an opioid effect, compounds with a weak, strong or extremely strong opioid effect, i.e. with a corresponding analgesic effect, may be used.

As compounds with a weak opioid effect, preferably codeine, dextropropoxyphene, dihydrocodeine, diphenoxylate, ethylmorphine, meptazinol, nalbuphine, pethidine (meperidine), tilidine, tramadol or viminol are used, as compounds with a strong opioid effect preferably butorphanol, dextromoramide, dezocine, diacetylmorphine (heroin), hydrocodone, hydromorphone, ketobemidone, levomethadone, levomethadyl acetate, levorphanol, morphine, nalorphine, oxycodone, pentazocine or piritramide are used and as compounds with an extremely strong opioid effect preferably alfentanil, buprenorphine, etorphine, fentanyl, remifentanil or sufentanil are used.

As a physiologically acceptable salt of the compound with an opioid effect, preferably hydrochloride, hydrobromide, sulphate, sulphonate, phosphate, tartrate, embonate, formate, acetate, propionate, benzoate, oxalate, succinate, citrate, glutamate, fumarate, aspartate, glutarate, stearate, butyrate, malonate, lactate, mesylate or a mixture of at least two of these salts are used.

In a particularly preferred embodiment of the invention, the new active substance combination contains, as a compound with an opioid effect, morphine or fentanyl and/or at least one corresponding physiologically acceptable salt.

Very particularly preferred, the new active substance combination contains as component A) morphine or fentanyl and as component B) a compound of the general formula I, with the group R standing for the group a) and the groups R¹, R², R³ each meaning H, and/or one of its enantiomers and/or one of its diastereomers and/or a corresponding physiologically acceptable salt.

In a further very particularly preferred embodiment, the inventive active substance combination contains as component A) morphine or fentanyl and as component B) a compound of the general formula I, with the group R standing for the group c), the group R¹ standing for CH₃ and the group R² meaning H, and/or one of its enantiomers and/or one of its diastereomers and/or a corresponding physiologically acceptable salt.

If the inventive active substance combination contains as component A) a compound with a weak opioid effect, the weight ratio of component B) to component A) should preferably be in the range from 1:0.02 to 1:10, particularly preferably in the range from 1:0.1 to 1:5 and very particularly preferably in the range from 1:0.5 to 1:2.5.

If component A) is a compound with a strong opioid effect, the weight ratio of component B) to component A) should preferably be in the range from 1:0.002 to 1:1, particularly preferably in the range from 1:0.005 to 1:0.5 and very particularly preferably in the range from 1:0.01 to 1:0.25.

For compounds with an extremely strong opioid effect as component A), the weight ratio of component B) to component A) in the inventive active substance combination should preferably be in the range from 1:0.0002 to 1:0.1, particularly preferably in the range from 1:0.0005 to 1:0.05 and very particularly preferably in the range from 1:0.001 to 1:0.025.

Another object of the invention is also medicaments containing the inventive active substance and possibly other active ingredients and/or inactive ingredients. Preferably, these inventive medicaments will be used to alleviate pain, particularly preferably to alleviate chronic and/or acute pain.

Another object of the invention is also pharmaceutical formulations in different pharmaceutical forms containing the inventive active substance combination and possibly other active substances and/or inactive substances.

Preferred pharmaceutical formulations are tablets, chewable tablets, chewing gums, dragees, capsules, drops, juices, syrups, suppositories, transmucal therapeutic systems, transdermal therapeutic systems, solutions, emulsions, suspensions, easily reconstitutable dry preparations, powders or sprays. Particularly preferred pharmaceutical formulations are tablets, capsules, drops or solutions.

In a further preferred embodiment, the inventive formulations are in multiparticulate form, preferably microtablets, microcapsules, microspheriods, ion-exchange resinates, granules, active substance crystals or pellets, particularly preferably as microtablets, granules or pellets. Pellets within the meaning of this invention also include pellets or built-up pellets produced by extrusion and/or spheronisation.

Preferably, the inventive pharmaceutical formulations are suitable for oral, intravenous, intramuscular, subcutaneous, intrathecal, epidural, buccal, sublingual, pulmonary, rectal, transdermal, nasal or intracerebroventricular application, with pharmaceutical formulations for oral or intravenous application being particularly preferred.

Suitable for oral application are preferably preparations in the form of tablets, chewable tablets, chewing gums, dragees, capsules, granules, drops, juices and syrups. Suitable for buccal application is preferably a transmucal therapeutic system. Suitable for parenteral, topical and inhalative application are preferably solutions, suspensions, emulsions, easily reconstitutable dry preparations, microspheroids, sprays, suppositories or plasters (e.g. transdermal therapeutic systems). Particularly preferred for parenteral application are suppositories or solutions, transdermal therapeutic systems for topical application and powders or solutions for inhalative application.

For the preparation of the inventive pharmaceutical formulations, in addition to an inventive active substance combination, preferably carrier materials, fillers, solvents, diluents, colorants, aromatics, binding agents or mixtures of at least two of these materials may be used. The choice of these inactive substances and their quantity depends upon the way in which the medicament is to be applied. A person skilled in the art is aware of the inactive substances suitable for the relevant form of application and their suitable quantities. The inventive pharmaceutical formulations may be produced in accordance with the conventional methods known to a person skilled in the art.

The inventive pharmaceutical formulations may also contain at least one of the active substance components A) or B) in a retarded form.

The retardation of the relevant active ingredient component is preferably performed by a retardant coating, by fixing to an ion-exchange resinate, by embedding in a retarding matrix or by a combination of these retardations. Suitable, retardant coatings include water-insoluble waxes or polymers, for example acrylic resins, preferably poly(meth)acrylate, or water-insoluble celluloses, preferably ethyl cellulose. These materials are known from prior art, e.g. Bauer, Lehmann, Osterwald, Rothgang, “Überzogene Arzneiformen” (Coated Drugs), Wissenschaftliche Verlagsgesellschaft mbH Stuttgart, 1988, p 69 et seq. These are listed here as references and hence count as part of the disclosure.

In addition to water-insoluble polymers, if necessary to set the release rate of the active substance in question, the retardant coatings may also contain non-retarding, preferably water-soluble polymers in quantities of up to 30 wt. %, such as polyvinylpyrrolidone or water-soluble celluloses, preferably hydroxypropyl methylcellulose or hydroxypropyl cellulose and/or hydrophilic pore formers, such as sucrose, sodium chloride or mannitol and/or the known plasticisers.

In addition, the inventive active substance combination may also have further coatings. Coatings may also include those which dissolve depending upon the pH. In this way it may be achieved that the pharmaceutical formulation passes through the gastric tract undissolved and the inventive active substance combination is only released in the intestinal tract. It is also possible to use coatings which serve to improve the taste.

Another common procedure for retardation is to fix the inactive substance to ion-exchange resins. Cation exchange resins, preferably polystyrene sulphonates, are used for the retardation of both the active substance component A) and the active substance component B).

For the retardation, the inventive active substance combination may also be present in a retarding matrix, preferably uniformly distributed. As matrix materials it is possible to use physiologically acceptable hydrophilic materials known to a person skilled in the art. Preferably, polymers, particularly preferably cellulose ethers, cellulose esters and/or acrylic resins are used as the hydrophilic matrix materials.

Very particularly preferred as matrix materials are ethyl cellulose, hydroxypropyl methylcellulose, hydroxypropyl cellulose, hydroxymethyl cellulose, poly(meth)acrylic acid and/or their derivatives, such as their salts, amides or esters.

Also preferred are matrix materials made of hydrophobic materials, such as hydrophobic polymers, waxes, fats, long-chain fatty acids, fatty alcohols or corresponding esters or ethers or mixtures thereof. Particularly preferred as hydrophobic materials are mono- or diglycerides of C₁₂-C₃₀ fatty acids and/or C₁₂-C₃₀ fatty alcohols and/or waxes or mixtures thereof.

It is also possible to use mixtures of the said hydrophilic and hydrophobic materials as a retarding matrix material.

In another preferred embodiment, the inventive pharmaceutical formulation contains at least one of the active substance components A) and B) in both their retarded form and their unretarded form. Through combination with the immediately released active substance it is possible to achieve a high initial dose for rapid pain relief. The slow release from the retarded form will then prevent any decrease of the analgesic effect.

The quantity of the inventive active substance combination to be administered to the patient varies, for example in dependence upon the weight of the patient, the method of application, the indication and the severity of the disease. Preferably, the quantity to be administered and the release of the inventive active substance combination should be set so that application is necessary a maximum of twice, preferably only once, a day.

With a single application a day, the inventive active substance combinations preferably contain 0.1 to 2000 mg, particularly preferably 0.5 mg to 1000 mg of the active substance component A) and preferably 0.1 to 100 mg, particularly preferably 0.5 to 50 mg of the active substance component B).

With two applications a day, the inventive active substance combinations preferably contain 0.05 to 1000 mg, particularly preferably 0.25 to 500 mg, of the active substance component A) and preferably 0.05 to 50 mg, particularly preferably 0.25 to 25 mg of the active substance component B).

Another object of the invention is also the use of an inventive active substance combination for the manufacture of a medicament, preferably for the manufacture of a medicament for the alleviation of pain, particularly preferably for the alleviation of chronic and/or acute pain, since the inventive active substance combination is very effective for the alleviation of severe to very severe pain, in particular for the alleviation of chronic and/or acute pain. The inventive active substance combination also surprisingly has a better analgesic efficacy compared to the administration of exclusively opioid active substances and has fewer undesirable side effects.

Advantageously, therefore, the dose of the compound with opioid efficacy required for effective pain alleviation may be reduced. The result of this is that the undesirable side effects which usually accompany the application of a compound of this type, such as respiratory depression, vomiting, dependence, sedation, constipation or tolerance development no longer occur or only occur in a very mild form.

Pharmacological Investigations

Tail-Flick-Test

The analgesic effect of the inventive active substance combinations and the comparative solutions was investigated in the focal-ray (tail-flick) test on rats according to D'Amour and Smith, J. Pharm. Exp. Ther. Vol. 72, pages 74-79, 1941. For this, female Sprague Dawley rats (Janvier, France) weighing between 120 and 150 g were used.

The rats were each placed in a test cage and the base of the tail exposed to the focused radiant heat from an electric lamp (tail-flick type 50/08/1.bc, Labtec, Dr. Hess). The lamp intensity was set so that the time from when the lamp was switched on until the tail was suddenly jerked away (pain latency) was 3 to 5 seconds in untreated rats. Before the application of the inventive active substance combinations and the comparative solutions, the rats were pre-tested twice within five minutes and the mean value of these measurements calculated as the pre-test mean value. The solutions of the inventive active substance combinations and the comparative solutions were then applied intravenously. The pain measurements were performed at 10, 20, 40 and 60 minutes respectively after the intravenous application. The analgesic effect was determined as an increase in the pain latency (% of the maximum possible antinociceptive effect) according to the following formula:

[(T₁−T₀)/(T₂−T₀)]×100

Here, the time T₀ is the latent time before the application, the time T₁ is the latent time after the application of the active substance combination and the time T₂ is the maximum exposure time (12 seconds).

The following examples are intended to explain the invention, but do not restrict the general inventive concept.

### EXAMPLES

### Example 1

To investigate the analgesic effect of the active substance combination containing as component A) morphine and as component B) the compound of the general formula I, with the group R standing for the group a) and the groups R¹, R², R³ each meaning H, each rat in a first group of 10 rats was intravenously given a 0.9% solution of sodium chloride containing 1.46 mg of morphine per kg of the rat's body weight and 21.5 mg of the said component B per kg of the rat's body weight.

### Comparative Example 1

For the comparison, each rat in a second group of 10 rats was given intravenously a 0.9% solution of common salt containing only 1.46 mg of morphine per kg of the rat's body weight.

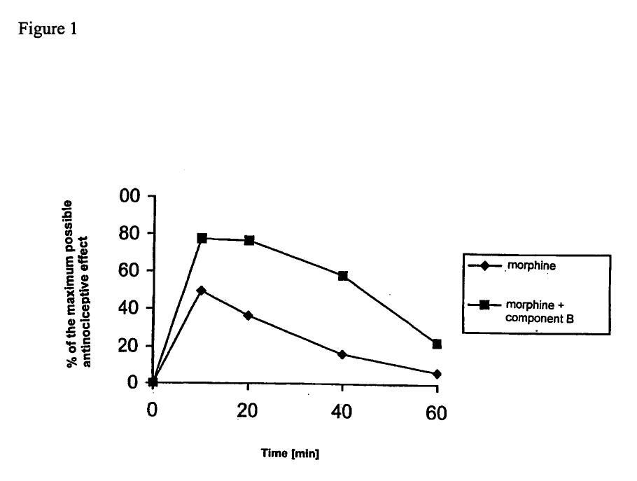

The results of these experiments are shown in FIG. 1.

### Example 2

To investigate the analgesic effect of an inventive substance combination containing as component A) morphine and as component B) the compound of the general formula I, with the group R standing for the group c), the group R¹ standing for CH₃ and the group R² meaning H, each rat in a third group of 10 rats was intravenously given a 0.9% solution containing 1.46 mg of morphine per kg of the rat's body weight and 46.4 mg of the said component B per kg of the rat's body weight.

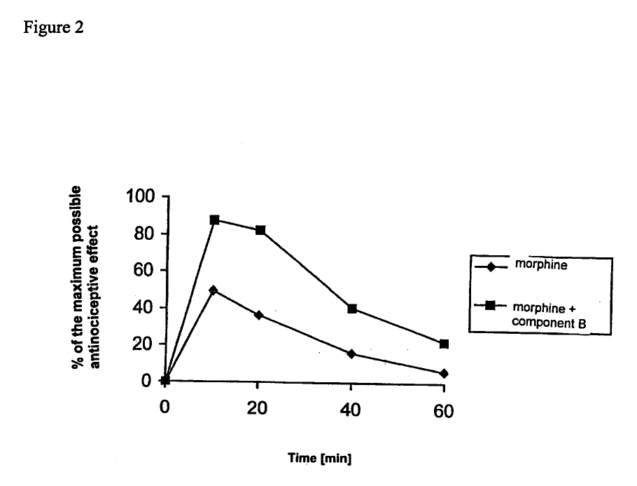

The results of this experiment are shown together with the result of the comparative solution according to comparative example 1 in FIG. 2. As is evident from FIGS. 1 and 2, the comparative solution according to the comparative example 1, which only contains morphine, already has a good analgesic effect.

As is also evident from FIGS. 1 and 2, the application of the solutions according to examples 1 and 2 containing the inventive active substance combinations of morphine and the said active substance component B) in each case has a greatly improved analgesic effect compared to the sole application of morphine.

1. Active substance combination containing
A) a compound with an opioid effect and/or at least one of its physiologically acceptable salts and
B) at least one compound of the general formula I 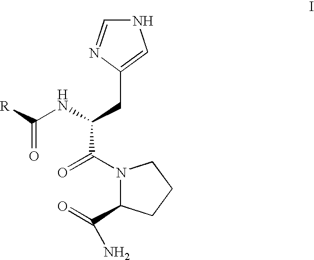
with the group R standing for one of the following groups a) to e) 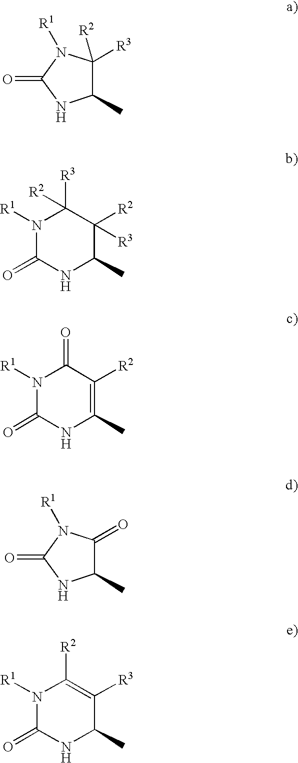
and the groups R¹, R², R³ each, the same or different, meaning an H or CH₃ group, and/or one of its enantiomers and/or one of its diastereomers and/or at least one corresponding physiologically acceptable salt.

2. Active substance combination according to claim 1, characterised in that the group R stands for the group a) and the groups R¹, R² and R³ each mean H.

3. Active substance combination according to claim 1, characterised in that the group R stands for the group c) and the group R¹ stands for CH₃ and the group R² means H.

4. Active substance combination according one of claims 1 to 3, characterised in that as component A), a compound with a weak, strong or very strong opioid effect is present.

5. Active substance combination according to claim 4, characterised in that as a compound with a weak opioid effect, codeine, dextropropoxyphene, dihydrocodeine, diphenoxylate, ethylmorphine, meptazinol, nalbuphine, pethidine (meperdine), tilidine, tramadol or viminol is present.

6. Active substance combination according to claim 5, characterised in that the weight ratio of component B) to component A) is in the range from 1:0.02 to 1:10, preferably in the range from 1:0.1 to 1:5, particularly preferably in the range from 1:0.5 to 1:2.5.

7. Active substance combination according to claim 4, characterised in that as a compound with a strong opioid effect, butorphanol, dextromoramide, dezocine, diacetylmorphine (heroin), hydrocodone, hydromorphone, ketobemidone, levomethadone, levomethadyl acetate, levorphanol, morphine, nalorphine, oxycodone, pentazocine or piritramide, preferably morphine, is present.

8. Active substance combination according to claim 7, characterised in that the weight ratio of component B) to component A) is in the range from 1:0.002 to 1:1, preferably in the range from 1:0.005 to 1:0.5, particularly preferably in the range from 1:0.01 to 1:0.25.

9. Active substance combination according to claim 4, characterised in that as a compound with a very strong opioid effect, alfentanil, buprenorphine, etorphine, fentanyl, remifentanil or sufentanil, preferably fentanyl, is present.

10. Active substance combination according to claim 9, characterised in that the weight ratio of component B) to component A) is in the range from 1:0.0002 to 1:0.1, preferably in the range from 1:0.0005 to 1:0.05, particularly preferably in the range from 1:0.001 to 1:0.025.

11. Active substance combination according to one of claims 1 to 10, characterised in that as a physiologically acceptable salt of the compound with an opioid effect, a hydrochloride, hydrobromide, sulphate, sulphonate, phosphate, tartrate, embonate, formate, acetate, propionate, benzoate, oxalate, succinate, citrate, glutamate, fumarate, aspartate, glutarate, stearate, butyrate, malonate, lactate, mesylate or a mixture of at least two of these salts is present.

12. Active substance combination according to one of claims 1 to 11, characterised in that as a physiologically acceptable salt of the compound of the general formula I and/or its enantiomers and/or its diastereomers, a hydrochloride, hydrobromide, sulphate, sulphonate, phosphate, tartrate, embonate, formate, acetate, propionate, benzoate, oxalate, succinate, citrate, glutamate, fumarate, aspartate, glutarate, stearate, butyrate, malonate, lactate, mesylate or a mixture of at least two of these salts is present.

13. Medicament containing an active substance combination according to one of claims 1 to 12 and optionally other active substances and/or inactive substances.

14. Medicament according to claim 13 for the alleviation of pain.

15. Medicament according to claim 14 for the alleviation of chronic pain.

16. Medicament according to claim 14 for the alleviation of acute pain.

17. Pharmaceutical formulation containing an active substance combination according to one of claims 1 to 12 and optionally other active substances and/or inactive substances.

18. Pharmaceutical formulation according to claim 17, characterised in that it is in the form of tablets, chewable tablets, chewing gums, dragees, capsules, suppositories, transdermal therapeutic systems, transmucal therapeutic systems, drops or as juice, syrup, solution, emulsion, suspension, easily reconstitutable dry preparations, powder or spray, preferably in the form of tablets, capsules, drops or solution.

19. Pharmaceutical formulation according to claim 17, characterised in that it is present in multiparticulate form, preferably microtablets, microcapsules, microspheriods, ion-exchange resonates, granules, active substance crystals or pellets, particularly preferably as microtablets, granules or pellets.

20. Pharmaceutical formulation according to one of claims 17 to 19, for oral, intravenous, intramuscular, subcutaneous, intrathecal, epidural, buccal, sublingual, rectal, pulmonary, transdermal, nasal or intracerebroventricular, preferably oral or intravenous, application.

21. Pharmaceutical formulation according to one of claims 17 to 20, characterised in that at least one of the active substance components A) or B) is present in retarded form.

22. Pharmaceutical formulation according to claim 21, characterised in that the retardation is performed by a retardant coating, by fixing to an ion-exchange resin, by embedding in a retarding matrix or by a combination thereof.

23. Pharmaceutical formulation according to claim 22, characterised in that that the coating is based on a water-insoluble polymer or wax.

24. Pharmaceutical formulation according to claim 23, characterised in that a poly(acrylic) resin or cellulose derivative, preferably alkyl cellulose, is used as a water-insoluble polymer.

25. Pharmaceutical formulation according to claim 24, characterised in that ethyl cellulose and/or a poly(meth)acrylate is used is used as a polymer.

26. Pharmaceutical formulation according to claim 22, characterised in that the matrix contains hydrophilic matrix material, preferably polymers, particularly preferably cellulose ethers, cellulose esters and/or acrylic resins, very particularly preferably ethyl cellulose, hydroxypropyl methylcellulose, hydroxypropyl cellulose, hydroxymethyl cellulose, poly(meth)acrylic acid and/or their salts, amides and/or esters.

27. Pharmaceutical formulation according to claim 22 or 26, characterised in that the matrix contains hydrophobic matrix material, preferably polymers, waxes, fats, long-chain fatty acids, fatty alcohols or corresponding esters or ethers or mixtures thereof, particularly preferably mono- or diglycerides of C₁₂-C₃₀ fatty acids and/or C₁₂-C₃₀ fatty alcohols and/or waxes or mixtures thereof.

28. Pharmaceutical formulation according to claim 22, characterised in that polystyrene sulphonates are used as cation-exchange resins.

29. Pharmaceutical formulation according to one or more of claims 21 to 28, characterised in that in addition to the retarded form, at least one of the active substance components A) or B) is present in the unretarded form.

30. Use of an active substance combination according to one of the claims 1 to 12 for the manufacture of a medicament.

31. Use according to claim 30 for the manufacture of a medicament for the alleviation of pain.

32. Use according to claim 31 for the manufacture of a medicament for the alleviation of chronic pain.

33. Use according to claim 31 for the manufacture of a medicament for the alleviation of acute pain.
